(19) United States
(12) Patent Application Publication
(10) Pub. No.: US 2009/0171453 A1
(43) Pub. Date: Jul. 2, 2009

(54) Drug Coated Stent Having a Surface Treatment and Method of Manufacturing

(76) Inventors: Tara K. Adams, Raleigh, NC (US); Jason S. Sturgeon, Spencer, IN (US); Manjiri A. Dhoke, Bloomington, IN (US); Dennis J. Delap, Bloomington, IN (US)

Correspondence Address: BRINKS HOFER GILSON & LIONE/CHICAGO/COOK, PO BOX 10395, CHICAGO, IL, 60610, US

(21) Appl. No.: 11/966,302

(22) Filed: Dec. 28, 2007

(51) Int. Cl. A61F 2/82

(52) U.S. Cl. 623/1.43; 623/1.42; 623/1.44

(57) ABSTRACT

Drug coated stents for implantation in a body vessel are provided. A stent can include a plasma treatment on the luminal surface and a protein-adherence deterring layer on at least a portion of the luminal surface. A further embodiment includes a method of manufacturing a drug coated stent. Another further embodiment includes a method of treating a vascular condition with a drug coated stent.

### BACKGROUND

The present invention relates to implantable medical devices medical devices and more particularly, to drug coated stents. The invention further provides methods of manufacturing drug coated stents having a surface treatment, and methods of treating a vascular condition with an implantable stent.

Stents are useful to address a number of medical conditions. Among other things, stents are used to treat atherosclerosis. Atherosclerosis may occur when cholesterol levels and/or scar tissue build up which causes the arteries to narrow and restrict blood flow. One type of procedure used to treat atherosclerosis is angioplasty, a non-invasive procedure that widens narrowed or blocked arteries. In one example of angioplasty, a catheter with a deflated balloon is inserted into the narrowed artery segment. The balloon is inflated to force open the artery, and then the catheter is deflated and withdrawn. Following angioplasty, a stent may be placed at a site where closure is to be prevented. A stent acts like a scaffold and will remain in the artery after the catheter is withdrawn to keep a vessel open.

Angioplasty may be followed by an abrupt closure of the vessel or by a more gradual closure of the vessel, commonly known as restenosis. Restenosis may result from the natural healing reaction to the injury to the vessel wall and the implantation of a medical device such as a stent. Restenosis may occur in the presence of a stent as tissue walls push into openings within the tubular surface of the stent or around the ends of a stent. Platelets, leukocytes, and other protein containing elements may attach to an implanted stent or the tissue surrounding an implanted stent. Intimal hyperplasia, which involves the migration and proliferation of medial smooth muscle cells may also occur and result in occlusion of a treated vessel.

One method that has been employed to prevent restenosis and a related problem, thrombosis, is the oral administration of drugs. Drugs may be given alone or in conjunction with stent treatment, and administered in conjunction with stent deployment. For example, drugs such as dextran, aspirin, or warfarin may be administered orally in conjunction with stent deployment to prevent restenosis and/or thrombosis. A drug may also be locally administered near the site of stent deployment with a device such as a dispatch catheter.

To address problems with restenosis and the inadequacy of previous methods stents have been developed that incorporate drugs and other substances. For example, U.S. Pat. No. 6,887,270 describes implantable multilayer tubular medical devices incorporating antimicrobial bioactive agents in a matrix polymer around a barrier layer lining the drainage lumen. The diameter of the drainage lumen can remain substantially constant, as the barrier layer can regulate the rate of release of the bioactive agent from the matrix polymer into the drainage lumen.

Drug coated stents are often designed to deliver therapy at a desired location or to provide a desired elution profile. For example, U.S. Pat. No. 6,908,622 to Barry et al. discloses a paclitaxel coated stent with a polymer coating that delivers a dose of paclitaxel with a specific elution profile.

The scientific literature also discloses that drugs coated on stents may produce distinct drug elution profiles. For example, Hwang et al: Physiological Transport Forces Govern Drug Distribution for Stent-Based Delivery, Circulation 2001; 104:600, discloses that hydrophobic drugs may have elution profiles that are different than elution profiles of hydrophilic drugs.

Nevertheless, research into elution profiles has failed to eliminate complications associated with drug coated stents. It has been recently reported that drug coated stents may cause an increase in thrombosis versus bare metal stents. See Trading Restenosis for Thrombosis? New Questions about Drug-Eluting Stents, New England Journal of Medicine 355; 19:1949 by Miriam Schuchman. Accordingly, solutions to the problem of restenosis that do not increase the risk of thrombosis are desirable.

An additional problem associated with stents coated in a non-biodegradable polymer is that the polymer coating may crack. Undesirable cracks may appear in a polymer coating particularly when a polymer is coated on the entire surface of a stent. Unexpected cracking can lead to unpredictable drug elution, and in severe cases, a piece of a nonbiodegradable polymer may become detached from the stent and cause an embolism. Cracking of polymer coatings may be increased in stents that are non-biodegradable and fully coated.

The prior art discloses certain attempts to prevent restenosis. For example, a method of modifying the surface of a medical device to make it capable of preventing restenosis has been disclosed. A method of plasma treatment is disclosed in U.S. Pat. No. 6,613,432 to Zamora, et al. According to Zamora, a metallic surface is treated with plasma comprising nitrogen-containing molecules and oxygen-containing molecules in which the molecules have less than seven atoms and preferably less than five atoms.

In addition, methods of modifying a surface of a medical device to make a surface bondable to a lubricious coating are also disclosed. For example, U.S. Application No. 2005/0008869 to Clark et al. discloses a method of surface modifying a fluoropolymer containing wire guide with a metallic sodium etchant. U.S. Pat. No. 5,356,433 to Rowland et al. discloses dipping stents in a solution comprising an amino-functional silane such as Silquist® Silane A-1110 (NH₂CH₂CH₂CH₂Si(OCH₃)₃) to provide a biocompatible surface.

A method of efficiently and inexpensively manufacturing drug coated stents also is desirable because the price of a coated stent may be significant. One estimate places the cost of a drug coated stent available on the market as much as five times the cost of a bare metal stent. Accordingly, efficient means of manufacturing drug-coated stents are desirable.

For the above mentioned reason and others not specifically mentioned, it is apparent to the inventor that stents with improved characteristics and methods of manufacturing drug coating stents are desirable.

### BRIEF SUMMARY

A stent device is provided wherein the luminal surface is treated and a protein-adherence deterring coating is attached to the treated surface. Preferably, the luminal surface is plasma treated or an organosilane is applied to the luminal surface. A hydrophobic drug is located on the abluminal surface of the stent. Addition details and advantages are further described below.

### BRIEF DESCRIPTION OF SEVERAL VIEWS OF THE DRAWINGS

The invention may be more fully understood by reading the following description in conjunction with the drawings, in which:

FIG. 1A is a side view of a drug coated stent

FIG. 1B is an end view of the stent shown in FIG. 1A

### DETAILED DESCRIPTION

### Definitions

As used herein the terms “comprise(s),” “include(s),” “having,” “has,” “contain(s),” and variants thereof, are intended to be open-ended transitional phrases, terms, or words that do not preclude the possibility of additional acts or structure.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel.

Unless other-wise indicated, as used herein, a “layer” refers to a portion of a structure having a defined composition or structure and a defined boundary with respect to an adjacent material.

The term “hydrophobic,” as used herein, refers to a substance with a solubility in water of less than 0.1 mg/mL at room temperature (about 25° C.).

The term “elution,” as used herein, refers to removal of a material from a substrate by means of an elution medium. The elution medium can remove the material from the substrate by any process, including by acting as a solvent with respect to the removable material. For example, in medical devices adapted for introduction to the vascular system, blood can act as an elution medium that dissolves a therapeutic agent releasably associated with a portion of the surface of the medical device. The removable material preferably includes the therapeutic agent, but can also include a bioabsorbable elastomer. The therapeutic agent can be selected to have a desired solubility in a particular elution medium. Unless otherwise indicated, the term “release” referring to the removal of the therapeutic agent from a coating in contact with an elution medium is intended to be synonymous with the term “elution” as defined above. Similarly, an “elution profile” is intended to be synonymous with a “release profile,” unless otherwise indicated.

An “elution medium,” as used herein, refers to a condition or environment that releases a therapeutic agent from a coating upon contact of the coating with the elution medium for a desired period of time. A suitable elution medium is any substance or environment into which the therapeutic agent can be released. The elution medium is desirably a fluid. More desirably, the elution medium is a biological fluid such as blood or porcine serum, although any other chemical substance can be used as an elution medium. For example, alternative elution media include phosphate buffered saline, blood, SDS, aqueous solutions, reaction conditions including temperature and/or pH, or combinations thereof, that release the therapeutic agent at a desired rate. Preferably, the elution medium is a fluid that provides an elution profile that is similar to the elution profile obtained upon implantation of the medical device within a body vessel. For example, porcine serum can provide an elution profile that is similar to the elution profile in blood for some coating configurations.

The term “luminal surface,” as used herein, refers to the portion of the surface area of a medical device defining at least a portion of an interior lumen. Conversely, the term “abluminal surface,” as used herein, refers to portions of the surface area of a medical device that do not define at least a portion of an interior lumen. For example, where the medical device is a tubular frame formed from a plurality of interconnected struts and bends defining a cylindrical lumen, the abluminal surface can include the exterior surface, sides and edges of the struts and bends, while the luminal surface can include the interior surface of the struts and bends.

As used herein, the term “body vessel” means any tube-shaped body passage lumen that conducts fluid, including but not limited to blood vessels such as those of the human vasculature system, billiary ducts and ureteral passages.

As used herein, the term “bioactive agent” as used herein refers to any pharmaceutically active agent that results in an intended therapeutic effect on the body to treat or prevent conditions or diseases. The terms “therapeutic agent” and “drug” may be taken to have the same meaning as “bioactive agent” and thus the terms may be used interchangeably.

Any concentration range, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers.

The term “alloy” refers to a substance composed of two or more metals or of a metal and a nonmetal intimately united, for example by chemical or physical interaction. Alloys can be formed by various methods, including being fused together and dissolving in each other when molten, although molten processing is not a requirement for a material to be within the scope of the term “alloy.” As understood in the art, an alloy will typically have physical or chemical properties that are different from its components.

The term “mixture” refers to a combination of two or more substances in which each substance retains its own chemical identity and properties.

Stent body

In a first embodiment, the present invention relates to implantable stents for placement within a body vessel. In general, the stent body has a cylindrical shape. A myriad of designs are known in the art for stents and all can be used in this invention.

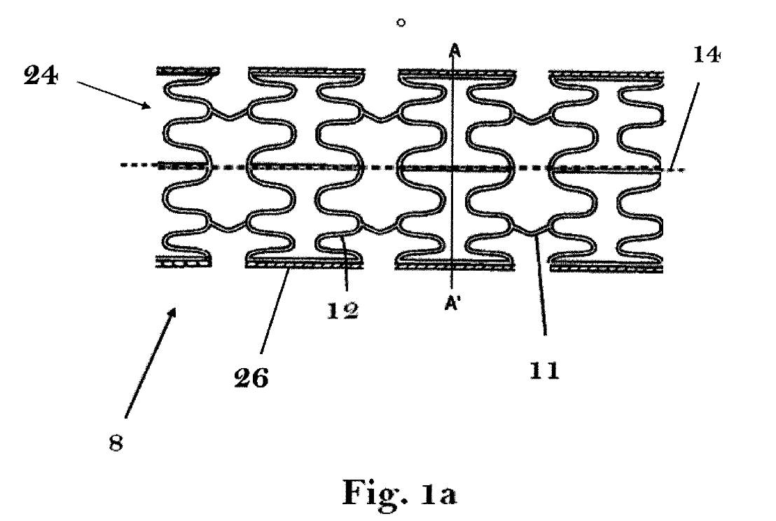

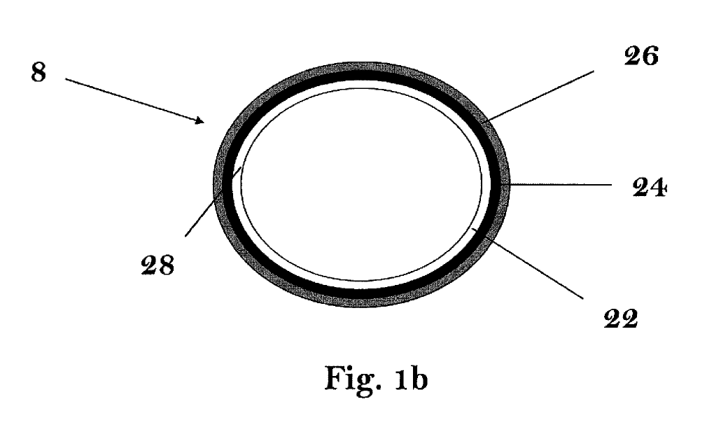

For example, in FIG. 1A, a stent 8 having a stent body 24 includes sinusoidal ring members 12 aligned around a longitudinal axis 14 to form a generally cylindrical or tubular shape. The sinusoidal ring members are joined by one or more longitudinal struts 11. In this embodiment, a hydrophobic drug layer 26 is shown on the abluminal surface of the stent 8. Line A-A′ bisects the stent 8 and forms a generally circular cross-section. Not shown in this view is a protein-adherence deterring (PAD) coating on the luminal surface of the stent 8.

FIG. 1B is an end view of the stent 8 shown in FIG. 1A. The stent body 24 is generally circular when the stent 8 is viewed from one end. A PAD coating 22 is attached to the luminal surface of the stent body 24. A hydrophobic drug layer 26 is attached to the abluminal surface of the stent body 24.

The stent body may comprise any material known in the art to be suitable for stents. For example, the stent body may comprise stainless steel, cobalt-chrome alloys, amorphous metals, tantalum, platinum, gold, titanium, or superelastic materials such as NITINOL. The stent may be a NITINOL stent that is commercially available such as a Z-STENT. Z-STENTS are available from Cook, Incorporated, Bloomington, Ind. USA.

In some embodiments, the stent body comprises a polymer material. The stent body may be entirely made of a polymer or polymers. Alternatively, the stent body may be only partially made of a polymer or polymers.

In one embodiment, the stent body comprises THORALON® THORALON® has been used as a component of stent bodies in certain vascular applications and is characterized by thromboresistance, high tensile strength, low water absorption, low critical surface tension, and good flex life. THORALON® is believed to be biostable and to be useful in vivo in long term blood contacting applications requiring biostability and leak resistance. Because of its flexibility, THORALON® is useful in procedures involving larger vessels where elasticity and compliance is beneficial.

In another embodiment, the stent body comprises a biocompatible polymer. For example, polyurethanes modified with cationic, anionic and aliphatic side chains may be used. See, for example, U.S. Pat. No. 5,017,664. Other biocompatible polyurethanes include: segmented polyurethanes, such as BIOSPAN; polycarbonate urethanes, such as BIONATE; and polyetherurethanes such as ELASTHANE; (all available from POLYMER TECHNOLOGY GROUP, Berkeley, Calif.). Other biocompatible polyurethanes include polyurethanes having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as BLAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL -AL, and CARBOSIL polymers are thermoplastic elastomer urethane copolymers containing siloxane in the soft segment, and the percent siloxane in the copolymer is referred to in the grade name. For example, PURSIL-10 contains 10% siloxane, These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

In addition, any of these biocompatible polyurethanes used in the stent body may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference

Protein Adherence Deterring Layer

According to certain embodiments, a PAD coating 22 is attached to at least a portion of the treated luminal surface. Preferably, the entire luminal surface of the stent is coated with a PAD coating.

The PAD coating desirably has at least two characteristics. First, it is hydrophilic so that when a hydrophobic drug is applied to the stent the hydrophobic drug does not easily adhere to the PAD coated surface, resulting in a higher concentration of hydrophobic drug on the abluminal surface compared to the luminal surface. Second, the PAD coating resists protein adhesion, resulting in a decrease in restenosis or thrombosis.

For example, a stent having a PAD coating comprising poly(ethylene)glycol attached to the luminal surface may be dipped in a solution of paclitaxel whereby the paclitaxel will attach primarily to the abluminal surface of the stent, and not to the luminal surface of the stent.

The PAD coating may be biodegradable or not biodegradable. When the PAD coating is biodegradable, the rate of dissolution can be varied by changing the molecular weights of the polymer serving as the PAD coating. Typically, the lower the molecular weight of a polymer, the faster the polymer will dissolve in an elution medium. The molecular weight of the polymer can be selected to provide a desired rate of dissolution and durability. Some polymers, such as chondroitin sulfate, may occur in nature with a molecular weight as high as 25,000, while others, such as hydroxypropylmethyl cellulose might be as high as 1,000,000. Hyaluronate may have a molecular weight as great as 8,000,000. When the PAD coating is biodegradable, the molecular weight of the polymer should be high enough so that the wet polymer has enough strength and film integrity remain intact during delivery of the medical device through a body vessel, and low enough so that the polymer may dissolve during a desired time frame following deployment of the medical device. Varying the thickness or adding perforations will also increase the rate of dissolution of the polymer.

The PAD coating comprises at least one polymer, and optionally may comprise two or more polymers. Preferably, the PAD coating comprises polyethylene or polyacrylic acid. For example, the PAD coating is most preferably a polyethyleneoxide or poly(ethylene glycol) (PEG), having a weight average molecular weight in the range from 100 to about 10,000. The PAD coating may include one or more esters of poly(meth)acrylic acid wherein the ester group may be represented by the formula —OR in which the R moiety is sufficiently small (e g., methyl or ethyl or other C1 or C2 type of moiety) so that the polymer is water soluble; similar esters of polyvinyl alcohol; combinations of these, and the like. Hyaluronate may also be used as a PAD coating.

The total amount of the PAD coating applied to the luminal surface of the graft material will depend on the composition of the stent. Preferably, an amount of PAD coating is applied that is sufficient to form a uniform intact coating on the luminal surface. Preferably the PAD coating has a thickness of about 0.5 to 1.0 μm.

Additives may also be added to affect desired properties of the PAD coating. For example, plasticizers are known in the art to increase flexibility when added to the removable material. Plasticizers include glycerol, sorbitol, polyethylene glycol, polypropylene glycol or sugars (glucose, maltodextrins, acetylated monoglycerides, citric or lactic acid esters). Brittleness may be reduced by adding esters of fatty acids and glycerol, examples of which include; glycerol monofatty acid esters, glycerol acetate fatty acid esters, glycerol lactate fatty acid esters, glycerol citrate fatty acid esters, glycerol succinate fatty acid esters, glycerol diacetyltartrate fatty acid esters and glycerol monoacetate. Other derivatives of the monoglyceride reacted with acetic acid, lactic acid, citric acid, succinic acid or diacetyltartaric acid may also be included in the ester of a fatty acid and glycerol.

Heparin, its derivatives, and related substances such a low molecular weight heparin may also be a suitable PAD coating. Heparin is a glycosaminoglycan with a molecular weight of between 3 kDA to 40 kDa. Herparin may also serve as a therapeutic agent that is incorporated within a separate PAD coating, or, in some embodiments, attached to the outside of a PAD coating.

Drug Layer

Preferably, the drug layer comprises a therapeutically effective amount of a hydrophobic drug or therapeutic agent on the abluminal surface of the stent. Additional hydrophobic drug or therapeutic agent also may be located elsewhere on the stent, for example, on the luminal surface of the stent, in wells or grooves on the stent, or in/on other layers such as the PAD layer or other abluminal layers.

The drug layer comprises at least one hydrophobic drug. The drug layer can also comprise additional drugs. The drug layer may contain a matrix, such as a polymer matrix. The polymer matrix may comprise a fill coating, which may provide an appropriate surface tension such that it dewets from the luminal surface.

Preferably, the hydrophobic drug is a taxane therapeutic agent. A taxane therapeutic composition includes paclitaxel (a compound of the chemical structure shown as structure (1) below) and derivatives thereof. Taxanes in general, and paclitaxel is particular, are taxane therapeutic compounds may function as a cell cycle inhibitor by acting as an anti-microtubule agent, and more specifically as a stabilizer. Preferred taxane therapeutic agents include the core structure with four fused rings (“core taxane structure,” shaded in structure (1)), with any therapeutically effective substituents.

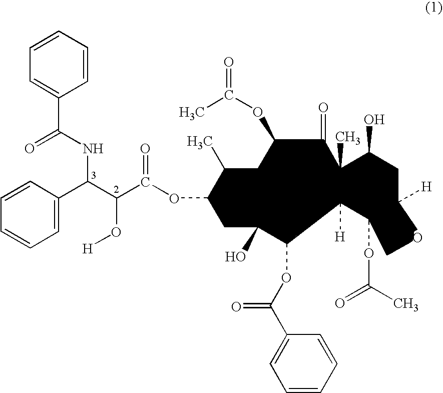

Paclitaxel has a molecular weight of about 853 amu, and may be readily prepared utilizing techniques known to those skilled in the art (see, e.g., Schiff et al., Nature 277: 665-667, 1979; Long and Fairchild, Cancer Research 54: 4355-4361, 1994; Ringel and Horwitz, J. Nat'l Cancer Inst. 83 (4): 288-291, 1991; Pazdur et al., Cancer Treat. Rev. 19 (4): 351-386, 1993; Tetrahedron Letters 35 (52): 9709-9712, 1994; J. Med. Chem. 35: 4230-4237, 1992; J. Med. Chem. 34: 992-998, 1991; J. Natural Prod. 57 (10): 1404-1410, 1994; J. Natural Prod. 57 (11): 1580-1583, 1994; J. Am, Chem. Soc. 110: 6558-6560, 1988), or obtained from a variety of commercial sources, including for example, Sigma Chemical Co., St. Louis, Mo. (T7402—from Taxus brevifolia).

Preferred taxane analogs and derivatives core vary the substituents attached to the core taxane structure. In one embodiment, the therapeutic agent is a taxane analog or derivative including the core taxane structure (1) and the methyl 3-(benzamido)-2-hydroxy-3-phenylpropanoate moiety (shown in structure (2) below) at the 13-carbon position (“C13”) of the core taxane structure (outlined with a dashed line in structure (1)).

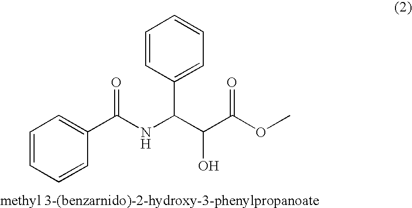

Compounds comprising structure (2) at the 13-carbon position of the core taxane structure may function to alter the biological activity of the taxane molecule as a cell cycle inhibitor. Examples of therapeutic agents having structure (2) include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

The drug coated stent may comprise a therapeutically effective amount of a hydrophobic rapamycin therapeutic agent. A rapamycin therapeutic agent includes rapamycin (a compound of the chemical structure shown as structure (3) below) and derivatives thereof. Rapamycin has a molecular weight of about 914 amu, and may be prepared utilizing techniques known to those skilled in the art or obtained from a variety of commercial sources such as Wyeth under the trade name Rapamune. Preferred rapamycin therapeutic agents include rapamycin and its derivatives such as everolimus.

In addition to the PAD coating, a therapeutic agent or drug may be in communication with the PAD coating. Preferably, the therapeutic agent is hydrophilic which may enhance adherence to the PAD. Preferably, the drug in communication with the PAD coating is a biodeposition-reducing bioactive agent. Preferably, the PAD coating comprises a biodegradable material and a bioactive agent selected to reduce or eliminate the adhesion of bacteria within the drainage lumen of the medical device. The bioactive agent preferably includes one or more antimicrobial agents. The term “antimicrobial agent” refers to a bioactive agent effective in the inhibition of, prevention of or protection against microorganisms such as bacteria, microbes, fungi, viruses, spores, yeasts, molds and others generally associated with infections such as those contracted from the use of the medical articles described herein. The antimicrobial agents include antibiotic agents and antifungal agents. Antibiotic agents include cephaloporins, clindamycin, chloramphenicol, carbapenems, penicillins, monobactams, quinolones, tetracycline, macrolides, sulfa antibiotics, trimethoprim, fusidic acid and aminoglycosides. Antifungal agents include amphotericin B, azoles, flucytosine, cilofungin and nikkomycin Z. Specific non-limiting examples of suitable antibiotic agents include: ciprofloxacin, doxycycline, amoxicillin, metronidazole, norfloxacin (optionally in combination with ursodeoxycholic acid), ciftazidime, and cefoxitin. Other suitable antibiotic agents include rifampin, minocycline, novobiocin and combinations thereof discussed in U.S. Pat. No. 5,217,493 (Raad et al.). Rifampin is a semisynthetic derivative of rifamycin B, a macrocyclic antibiotic compound produced by the mold Streptomyces mediterranic. Rifampin is believed to inhibit bacterial DNA-dependent RNA polymerase activity and is bactericidal in nature. Rifampin is available in the United States from Merrill Dow Pharmaceuticals, Cincinnati, Ohio. Minocycline is a semisynthetic antibiotic derived from tetracycline. It is primarily bacteriostatic and is believed to exert an antimicrobial effect by inhibiting protein synthesis. Minocycline is commercially available as the hydrochloride salt which occurs as a yellow, crystalline powder and is soluble in water and slightly soluble in alcohol. Minocycline is available from Lederle Laboratories Division, American Cyanamid Company, Pearl River, N.Y. Novobiocin is an antibiotic obtained from cultures of Streptomyces niveus or S. spheroides. Novobiocin is usually bacteriostatic in action and is believed to interfere with bacterial cell wall synthesis and inhibit bacterial protein and nucleic acid synthesis. Novobiocin also appears to affect stability of the cell membrane by complexing with magnesium. Novobiocin is available from The Upjohn Company, Kalamazoo, Mich.

Bactericidal nitrofuran compounds, such as those described by U.S. Pat. No. 5,599,321 (Conway et al.), incorporated herein by reference, can also be used as an antimicrobial bioactive agent. Preferred nitrofuran bioactive agents include nitrofurantoin, nitrofurazone, nidroxyzone, nifuradene, furazolidone, furaltidone, nifuroxime, nihydrazone, nitrovin, nifurpirinol, nifurprazine, nifuraldezone, nifuratel, nifuroxazide, urfadyn, nifurtimox, triafur, nifurtoinol, nifurzide, nifurfoline, nifuroquine, and derivatives of the same, and other like nitrofurans which are both soluble in water and possess antibacterial activity. References to each of the above cited nitrofuran compounds may be found in the Merck Index, specifically the ninth edition (1976) and the eleventh edition (1989) thereof, published by Merck & Co., Inc., Rahway, N.J., the disclosures of which are each incorporated herein by reference.

The antimicrobial agent can also comprise nanosize particles of metallic silver or an alloy of silver containing about 2.5 wt % copper (hereinafter referred to as “silver-copper”), salts such as silver citrate, silver acetate, silver benzoate, bismuth pyrithione, zinc pyrithione, zinc percarbonates, zinc perborates, bismuth salts, various food preservatives such as methyl, ethyl, propyl, butyl, and octyl benzoic acid esters (generally referred to as parabens), citric acid, benzalkonium chloride (BZC), rifamycin and sodium percarbonate.

Another example of a suitable antimicrobial agent is described in published U.S. patent application US2005/0008763A1 (filed Sep. 23, 2003 by Schachter), incorporated herein by reference. The PAD coating can be combined with a siloxane binder and divalent metallic (M²⁺) ions, such as, for example, Cu²⁺, Zn²⁺, Ca²⁺, Co²⁺, and Mn²⁺. Upon curing, the siloxane binder can form a silsesquioxane, e.g., methyl silane sesquioxide or CH₃SiO_{3/2}. The siloxane oligomeric binder can be synthesized, for example by hydrolysis of precursors such as, for instance, monomethylalkoxysilane, e. g., methyltrimethoxysilane (CH₃Si(OCH₃)₃) to form a partial condensate of methyl trisilanol. The monomethylalkoxysilane also can be provided in a mixture with copolymerizable silane monomer(s). A copolymer may be formed from cohydrolyzed silanol, RSi(OH)₃, of which methyl trisilanol comprises at least about 70% by weight, preferably at least about 75% by weight, and wherein R is a non-reactive organic moiety, such as, for example, e.g., lower alkyl (e g., C₁-C₆ alkyl, especially C₁-C₃ alkyl, methyl, ethyl or n- or iso-propyl), vinyl, 3,3,3-trifluoropropyl, γ-glycidyloxypropy, γ-methacryloxypropyl, and phenyl. When only methyl silanol (from methyl trialkoxysilane) is used, the amount of metal cation, (M²⁺) added can be based on the amount of silanol. When mixtures of silanol are used, the molar silane sesquioxide equivalent of the remaining silane mixture can be converted to the molar equivalent of methyl silane sesquioxide. In one example, the composition includes, on a weight basis of the total composition, from about 28% to 71%, preferably from about 31% to 71% silanol (of which at least about 70% is methylsilanol), from about 29% to about 39% water, from 0 to about 31%, preferably from about 15 to about 30%, isopropanol or other volatile organic solvent, and an M²⁺ ion or a mixture of such M²⁺ ions, within the range of from about 0.5 to 3 millimoles (gram×millimoles), preferably about 1.2 to 2.4 millimoles, per molar equivalent of the partial condensate calculated as methyl silane sesquioxide. The pH of the mixture is adjusted to mildly to slightly acidic, such as between 2.5 and 6.2, preferably 2.8 to 6.0, more preferably 3.0 to 6.0. More particularly, the aqueous coating composition can include a dispersion of divalent metal cations (such as Ca²⁺, Mn²⁺, Cu²⁺, and Zn²⁺) in a solution of water/lower aliphatic alcohol comprising the partial condensate of at least one silanol of the formula RSi(OH)₃ in which R is a moiety selected from the group consisting of lower alkyd vinyl, phenyl, 3,3,3-trifluoropropyl, γ-glycidyloxypropyl and γ-methacryloxypropyl. At least about 70 weight percent of the silanol in the solution is preferably CH₃Si(OH)₃. The solution further contains acid in an amount sufficient to provide a pH in the range of from about 2.5 to about 6.2. The divalent cations are included in the solution in an amount of from about 1.2 millimoles to about 2.4 millimoles per molar equivalent of the partial condensate, calculated as methyl silane sesquioxide.

In certain embodiments, materials with antimicrobial properties can be mixed with, or applied to, the interior surface of the PAD coating, or can be positioned between the PAD coating and the stent body, or within the stent body.

Methods of Manufacturing

In a further embodiment, methods for manufacturing a stent for placement within a body passage are provided. According to one method, a stent is provided having a luminal surface and an abluminal surface, At least a portion of the luminal surface may be treated to make it more adhesive to a protein-adherence deterring coating. A protein-adherence deterring coating is attached to at least a portion of the luminal surface and a hydrophobic drug is attached to at least a portion of the outer surface.

According to one preferred method, the luminal surface of a metallic stent is treated by plasma treatment to prepare the luminal surface for attachment of a PAD coating. A PAD coating is attached to the luminal surface of the stent.

Protein adhesion-deterring coatings tend not to adhere well to either metallic or polymer surfaces. Thus, it is preferable to treat select portions of these stents in order to increase adherence of the protein adhesion-deterring coating.

In embodiments of the invention, the luminal surface to be treated may include stainless steel, nitinol, titanium, or other metal alloys. The luminal surface to be treated may also include a polymer that could be a surface treated to provide improved adhesion of a PAD coating. For example, some polymer coatings such as poly tetrafluoroethylene (PTFE) and THORALON® may be relatively inert and may resist binding of a PAD coating.

Any suitable method known in the art may be employed to increase adherence of the PAD coating to a portion of the luminal surface of the stent. For example, the surface may be treated by ultrasonic spraying, misting, dipping, spreading, corona treatment, plasma treatment, or laser treatment. For further example, an organosilane may be applied to the luminal surface of the stent.

The surface treatment is preferably tailored to the stent composition. For example, metallic stents are preferably treated with a method that allows effective and efficient attachment of a PAD coating, such as plasma treatment, a directed ion treatment, or by applying an organosilane. Organosilanes are well-known in the art, and may be applied to the luminal surface of the stent to provide for attachment of the PAD coating. However, any surface treatment known in the art may be used.

After the treatment of the luminal surface with an organosilane, a coupling agent may be applied. For example, a coupling agent such as 1,3ethyledimethyl-aminopropyl carbodiimide (EDC) may be used. A coupling agent may be used to improve attachment of a therapeutic agent to the treated surface.

When the stent comprises a particularly inert surface, such as certain polymer materials, a preferable surface treatment is a chemical etchant. In one method of treatment, at least a portion of the luminal surface of a stent comprising a relatively chemically inert polymer may be treated with a solution comprising metallic sodium. For example, a metallic sodium solution such as FluoroEtch®, available from Acton Technologies, is effective for modifying the surface of certain polymers, such as PTFE. The solution may be applied in any manner known in the art.

Some methods of surface treatments may be easily directed to treat only the luminal surface, For instance, the luminal surface may be exposed to a mister which may be directed to only the inside of a tubular stent. Another method that is preferred due to its ability to be directionally controlled is an electrochemical method, such as plasma treatment. In cases where a method of surface modification is not easily directionally controlled, selected surfaces may be masked to prevent the surface treatment from affecting the surface not to be treated. Selectively masking the abluminal surface of a stent and then dipping or spraying the masked stent is a preferred method of surface treating a stent.

The surface treatment may also be applied to a stent before it is in a generally tubular conformation. For example, the bends and struts of a stent may be in a substantially flat conformation when a surface treatment is applied.

After the PAD coating is applied, a hydrophobic drug (or drugs) is applied to the abluminal surface of the stent. For example, the hydrophobic drug may be applied by immersing the stent in a solution comprising a hydrophobic drug. The hydrophobic drug will preferentially attach to the luminal surface of the stent as opposed to the abluminal surface because the hydrophilic PAD coating will resist adherence of the hydrophobic drug. The resulting stent will have a hydrophobic drug attached primarily to the abluminal surface and a hydrophilic PAD coating on the luminal surface that resists protein adherence.

Preferably, a PAD coating is applied to the abluminal surface of the stent graft using ultrasonic coating, to provide a smooth and uniform polymer coating. In one embodiment, PEG is preferably applied from an ultrasonic nozzle. A solution of 8 g/L PEG in a suitable solvent such as dichloromethane can be applied using an ultrasonic nozzle. Ultrasonic nozzles can be configured such that excitation of the piezoelectric crystals creates a transverse standing wave along the length of the nozzle. The ultrasonic energy originating from the crystals located in the large diameter of the nozzle body undergoes a step transition and amplification as the standing wave as it traverses the length of the nozzle. The ultrasonic nozzle can be designed so that a nodal plane is located between the crystals. For ultrasonic energy to be effective for atomization, the atomizing surface (nozzle tip) is preferably located at an anti-node, where the vibration amplitude is greatest. To accomplish this, the nozzle's length must be a multiple of a half-wavelength. Since wavelength is dependent upon operating frequency, nozzle dimensions can be related to operational frequency. In general, high frequency nozzles are smaller, create smaller drops, and consequently have smaller maximum flow capacity than nozzles that operate at lower frequencies. The ultrasonic nozzle can be operated at any suitable frequency, including 24 kHz, 35 kHz, 48 kHz, 60 kHz, 120 kHz or higher. Preferably, a frequency of 60-120 kHz or higher is used to atomize the solution of the bioabsorbable elastomer to the greatest possible extent so as to promote the formation of a smooth, uniform coating. Power can be controlled by adjusting the output level on the power supply. The nozzle power can be set at any suitable level, but is preferably about 0.9-1.2 W and more preferably about 1.0-1.1 W. The nozzle body can be fabricated from any suitable material, including titanium because of its good acoustical properties, high tensile strength, and excellent corrosion resistance. Liquid introduced onto the atomizing surface through a large, non-clogging feed tube running the length of the nozzle absorbs some of the vibrational energy, setting up wave motion in the liquid on the surface. For the liquid to atomize, the vibrational amplitude of the atomizing surface can be maintained within a band of input power to produce the nozzle's characteristic fine, low velocity mist. Since the atomization mechanism relies only on liquid being introduced onto the atomizing surface, the rate at which liquid is atomized depends largely on the rate at which it is delivered to the surface. Therefore, an ultrasonic nozzle can have a wide flow rate range. The maximum flow rate and median drop diameter corresponding to particular nozzle designs can be selected as design parameters by one skilled in the art. Alternatively, a PAD coating can be dissolved in a solvent(s) and sprayed onto the medical device using a conventional spray gun such as a spray gun manufactured by Badger (Model No. 200), an electrostatic spray gun, or most preferably an ultrasonic nozzle spray gun.

The PEG or other polymer can be dissolved in a solvent(s) and sprayed onto the medical device under a fume hood using a conventional spray gun, such as a spray gun manufactured by Badger (Model No. 200), or a 780 series spray dispense valve (EFD, East Providence, R.I.). Alignment of the spray gun and stent may be achieved with the use of a laser beam, which may be used as a guide when passing the spray gun over the medical device(s) being coated.

The distance between the spray nozzle and the stent/substrate can be selected depending on parameters apparent to one of ordinary skill in the art, including the area being coated, the desired thickness of the coating and the rate of deposition. Any suitable distance and nozzle size can be selected. For example, for coating an 80 mm long stent, a distance of between about 1-7 inches between the nozzle and stent is preferred, depending on the size of the spray pattern desired. The nozzle diameter can be, for example, between about 0.014-inch to about 0.046-inch.

Therapeutic agent may be applied to a surface of a medical device using a spray gun. The surface of the stent can be bare, surface modified, or have a primer coating previously applied to the medical device. Preferably, the coating applied to the surface consists essentially of the taxane therapeutic agent, and is substantially free of polymers or other materials.

Varying parameters in the spray coating process can result in different solid forms of the taxane therapeutic agent in a deposited coating. Spray coating parameters such as solvent system, fluid pressure (i.e., tank pressure), atomization pressure, ambient temperature and humidity. The solvent is desirably volatile enough to be readily removed from the coating during or after the spray coating process, and is preferably selected from the solvents discussed with respect to the first embodiment for each solid form of a taxane therapeutic agent.

Methods of Treatment

In another embodiment, the invention relates to methods of treating a vascular condition. According to embodiments, a stent is provided wherein at least a portion of the luminal surface is treated to make it more capable of attaching a PAD coating to the treated surface. A hydrophobic drug is attached to a portion of the abluminal surface. According to traditional methods of implanting stents in body vessels, a stent is implanted within a body vessel.

While preferred embodiments of the invention have been described, it should be understood that the invention is not so limited, and modifications may be made without departing from the invention. The scope of the invention is defined by the appended claims, and all devices and methods that come within the meaning of the claims, either literally or by equivalence, are intended to be embraced therein.

We claim:

1. A stent comprising:
a stent body comprising a plasma treated luminal surface and an abluminal surface;
a protein-adherence deterring layer on at least a portion of the luminal surface; and
a hydrophobic drug layer on at least a portion of the abluminal surface.

2. The stent according to claim 1, wherein the protein-adherence deterring layer comprises poly(ethylene)glycol.

3. The stent according to claim 1, wherein the protein-adherence deterring layer comprises heparin.

4. The stent according to claim 1, wherein the stent body comprises metal.

5. The stent according to claim 1, wherein the stent body comprises a biocompatible polymer.

6. The stent according to claim 3, wherein the biocompatible polymer is a polyurethane urea.

7. The stent according to claim 1, wherein the hydrophobic drug layer comprises a taxane therapeutic agent.

8. The stent according to claim 1, wherein the protein-adherence deterring layer further comprises a bioactive agent.

9. The stent according to claim 1, wherein the protein-adherence deterring layer further comprises the hydrophobic drug.

10. The stent according to claim 1, further comprises an organosilane layer in communication with the luminal surface of the stent.

11. The stent according to claim 10, wherein said organosilane layer is disposed between the luminal surface of the stent and the protein-adherence deterring layer.

12. A method of manufacturing a drug coated stent comprising,
providing a stent body having a luminal surface and an abluminal surface;
treating at least a portion of the luminal surface to make it more adhesive to a protein-adherence deterring coating;
attaching a protein-adherence deterring coating to the at least a portion of the luminal surface;
attaching a hydrophobic drug layer to at least a portion of the abluminal surface.

13. The method of claim 12, further comprising the step of attaching a therapeutic agent to the outer surface of the protein-adherence deterring coating.

14. The method of claim 12, wherein a therapeutic agent is incorporated in the protein-adherence deterring coating.

15. The method of claim 12, wherein the step of treating at least a portion of the luminal surface is with plasma.

16. The method of claim 12, wherein the step of treating at least a portion of the luminal surface is with an organosilane.

17. The method of claim 12, wherein the protein-adherence deterring coating is polyethylene glycol.

18. The method of claim 12, wherein the hydrophobic drug is a taxane therapeutic agent.

19. A method of treating a vascular condition comprising the steps of:
providing a stent having a luminal surface and an abluminal surface; wherein at least a portion of the luminal surface is treated to improve attachment of a protein-adherence deterring coating to the treated surface, the protein-adherence deterring coating being attached to the at least a portion of the luminal surface, and a hydrophobic drug being attached to at least a portion of the abluminal surface; and
implanting the stent within a body vessel.

20. The method of claim 19, wherein the hydrophobic drug comprises a taxane therapeutic agent.
